(19) United States
(12) Patent Application Publication
(10) Pub. No.: US 2026/0113607 A1
(43) Pub. Date: Apr. 23, 2026

(54) PHYSIOLOGICAL SIGNAL SENSING DEVICE AND ACTIVATION METHOD THEREOF

(71) Applicant: Bionime Corporation, Taichung City (TW)

(72) Inventors: Chun-Mu Huang, Taichung City (TW); Chieh-Hsing Chen, Taichung City (TW)

(73) Assignee: Bionime Corporation, Taichung City (TW)

(21) Appl. No.: 19/355,536

(22) Filed: Oct. 10, 2025

(30) Foreign Application Priority Data
Oct. 18, 2024 (TW) 113139822

(51) Int. Cl. H04W 4/70; A61B 5/00; A61B 5/145; A61B 50/30

(52) U.S. Cl. CPC H04W 4/70; A61B 5/002; A61B 5/14503; A61B 5/14546; A61B 50/30

(57) ABSTRACT

A method for activating a physiological signal sensing device through a handheld device to measure a physiological signal of an analyte in biological fluids is provided. The physiological signal sensing device includes a transmitter including a power unit, a switch unit, a magnetic field sensing unit and a processing unit. The method includes: establishing an inductive magnetic field between the handheld device and the transmitter, causing the magnetic field sensing unit to generate an inductive signal; in a first operating cycle, activating the switch unit by the inductive signal, causing the power unit to connect to the processing unit to activate the processing unit; providing an activation signal by the processing unit to turn on the switch unit; causing the switch unit to enter a self-maintaining state; and in a second operating cycle, connecting the power unit to the processing unit through the switch unit in the self-maintaining state.

### CROSS-REFERENCE TO RELATED APPLICATION AND CLAIM OF PRIORITY

This application claims the benefit of Taiwan Patent Application No. 113139822, filed on Oct. 18, 2024, at the Taiwan Intellectual Property Office, the disclosures of which are incorporated herein in their entirety by reference.

### FIELD OF THE INVENTION

The present invention relates to a physiological signal sensing device and an activation method thereof, particularly a physiological signal sensing device activated using near field communication (NFC) technology. After activation, the physiological signal sensing device can maintain a self-maintaining state for an extended period to continuously measure physiological signals and transmit real-time measurement results to an external signal receiving device.

### BACKGROUND OF THE INVENTION

Diabetes is a major global public health problem with a continuously increasing prevalence, causing significant impacts on health and the economy. Although modern medicine has made significant progress in diabetes management, more attention and efforts are still needed for the prevention of diabetes and the control of blood glucose. Therefore, early diagnosis and timely treatment are crucial.

Diabetic patients often need to take long-term medication and monitor their blood glucose levels regularly. Therefore, effective and convenient blood glucose monitoring is the key to maintain stable blood glucose levels. Compared to traditional discrete blood glucose monitoring methods (such as finger-prick tests), continuous glucose monitoring (CGM) systems reduce the frequent fingertip blood pricking, which reduces pain and inconvenience, especially for patients who need frequent monitoring. Most importantly, the continuous glucose monitoring systems offer many advantages that traditional discrete methods cannot achieve, such as continuous monitoring, blood glucose trend analysis, high/low blood glucose alert systems, and automatic data recording.

Therefore, there is an urgent need to provide a physiological signal sensing device that is easy for users to operate, simplifying the activation and installation steps to optimize the user experience.

It is therefore the Applicant's attempt to deal with the above situations encountered in the prior art.

### SUMMARY OF THE INVENTION

One objective of the present invention is to provide a physiological signal sensing device activated by a handheld device using NFC technology. After activation, the physiological signal sensing device can maintain a self-maintaining state for an extended period to continuously measure physiological signals and transmit real-time measurement results to the external handheld device, and thus functions such as continuous monitoring, trend analysis, anomaly alerts, and automatic data recording are achieved.

In accordance with another aspect of the present disclosure, a physiological signal sensing device activated through a handheld device is disclosed. The physiological signal sensing device includes a sensor configured to be partially implanted subcutaneously to measure a physiological signal of an analyte in a biological fluid; and a transmitter coupled to the sensor. The transmitter includes a magnetic field sensing unit capable of generating inductive magnetic field with the handheld device at a predetermined distance to generate an inductive signal; a switch unit connected to the magnetic field sensing unit; a power unit connected to the switch unit; a processing unit connected to the switch unit and configured to receive and process the physiological signal measured by the sensor to generate a processed signal; and an antenna unit connected to the processing unit and configured to receive the processed signal and transmit the processed signal to the handheld device. In a first operating cycle, the switch unit is activated by the inductive signal, causing the power unit to connect to the processing unit through the switch unit, thereby activating the processing unit to provide an activation signal to turn on the switch unit, causing the switch unit to enter a self-maintaining state through the activation signal. In a second operating cycle, the power unit connects to the processing unit through the switch unit in the self-maintaining state to supply power to the processing unit.

In accordance with one more aspect of the present disclosure, a method for activating a physiological signal sensing device through a handheld device is disclosed. The physiological signal sensing device is configured to be partially implanted subcutaneously to measure a physiological signal of an analyte in a biological fluid and comprises a transmitter and a sensor, and the transmitter comprises a power unit, a switch unit, a magnetic field sensing unit and a processing unit. The method includes: establishing an inductive magnetic field between the handheld device and the transmitter at a predetermined distance, causing the magnetic field sensing unit to generate an inductive signal through the inductive magnetic field; in a first operating cycle, activating the switch unit by the inductive signal, causing the power unit to connect to the processing unit through the switch unit, thereby activating the processing unit; providing an activation signal by the processing unit to turn on the switch unit; causing the switch unit to enter a self-maintaining state through the activation signal; and in a second operating cycle, connecting the power unit to the processing unit through the switch unit in the self-maintaining state to supply power to the processing unit.

In accordance with one more aspect of the present disclosure, a method for activating a physiological signal sensing device through a handheld device is disclosed. The physiological signal sensing device is configured to be partially implanted subcutaneously to measure a physiological signal of an analyte in a biological fluid and comprises a transmitter and a sensor, and the transmitter comprises a power unit, a switch unit, a magnetic field sensing unit and a processing unit. The method includes: scanning a barcode tag of the transmitter using the handheld device to obtain access information of the transmitter; scanning a barcode tag of the sensor using the handheld device to obtain access information of the sensor; establishing an inductive magnetic field between the handheld device and the transmitter at a predetermined distance, causing the magnetic field sensing unit to generate an inductive signal through the inductive magnetic field; in a first operating cycle, turning on the switch unit by the inductive signal, causing the power unit to connect to the processing unit through the switch unit to activate the processing unit; providing an activation signal by the processing unit to turn on the switch unit; causing the switch unit to enter a self-maintaining state through the activation signal; and in a second operating cycle, connecting the power unit to the processing unit through the switch unit in the self-maintaining state to supply power to the processing unit.

In summary, a physiological signal sensing device that is easy for users to operate, simplifying the activation and installation steps to optimize the user experience is provided in the present invention.

### BRIEF DESCRIPTION OF THE DRAWINGS

Other objectives, advantages and efficacies of the present invention will be described in detail below taken from the preferred embodiments with reference to the accompanying drawings.

FIG. 1A is a schematic diagram of a handheld device and a physiological signal sensing device according to a specific embodiment of the present invention.

FIG. 1B is a schematic diagram of a usage scenario of the handheld device and the physiological signal sensing device according to the specific embodiment of the present invention.

FIG. 2 is a schematic diagram of retrieving data through an information tag according to a specific embodiment of the present invention.

FIG. 3A is a circuit diagram of the handheld device and a transmitter according to a specific embodiment of the present invention.

FIG. 3B is a schematic diagram of timing state changes of each electronic parameter signal in the specific embodiment of FIG. 3A.

FIG. 4A is a system function block diagram of a handheld device and a physiological signal sensing device according to another specific embodiment of the present invention.

FIG. 4B is a schematic diagram of timing state changes of each electronic parameter signal in another specific embodiment of FIG. 4A.

FIG. 5 is a flow chart of a method for activating the physiological signal sensing device according the specific embodiment of the present invention.

### DETAILED DESCRIPTION OF THE PREFERRED EMBODIMENTS

The present invention will now be described more specifically with reference to the following embodiments. It is to be noted that the following descriptions of preferred embodiments of this invention are presented herein for purpose of illustration and description only; they are not intended to be exhaustive or to be limited to the precise form disclosed. In the preferred embodiments, the same reference numeral represents the same element in each embodiment.

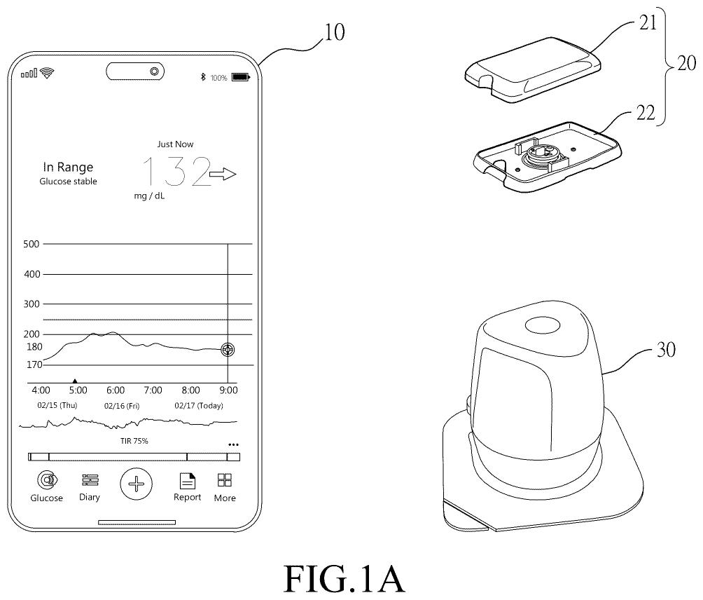

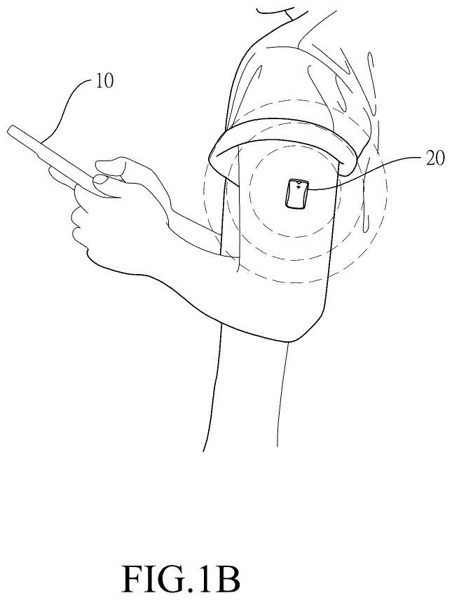

Please refer to FIG. 1A and FIG. 1B. The continuous glucose monitoring systems of the present invention includes a handheld device 10, a physiological signal sensing device 20 and an implanting device 30. The handheld device 10 can be a smartphone or a receiver with signal processing functions, and can have an interface for operation and display information related to the user's glucose or blood glucose levels. The physiological signal sensing device 20 may include a transmitter 21 and a sensor 22, wherein the sensor 22 is initially placed inside the implanting device 30 at the time of manufacturing. When in use, the sensor 22 is first implanted on the epidermis of a user through the implanting device 30, thereby a flexible needle is inserted into the subcutaneous tissue of the user's arm. The transmitter 21 is then mounted on the sensor 22 to wirelessly transmit analyte values measured by the sensor 22 to the handheld device 10. The user can obtain the blood glucose-related information (such as blood glucose trend, current blood glucose levels, historical blood glucose levels, etc.) through the user interface of the handheld device 10 to monitor the blood glucose over a full cycle. By monitoring the blood glucose level around the clock, the handheld device 10 can provide real-time alerts when the blood glucose level of the user is too low or too high. For example, when the user is in a hypoglycemic or hyperglycemic state, an alert window may be displayed on the user interface, or different alarm sounds may be used to indicate the hypoglycemic or hyperglycemic state. Alternatively, different vibration frequencies may be used to indicate the hypoglycemic or hyperglycemic state.

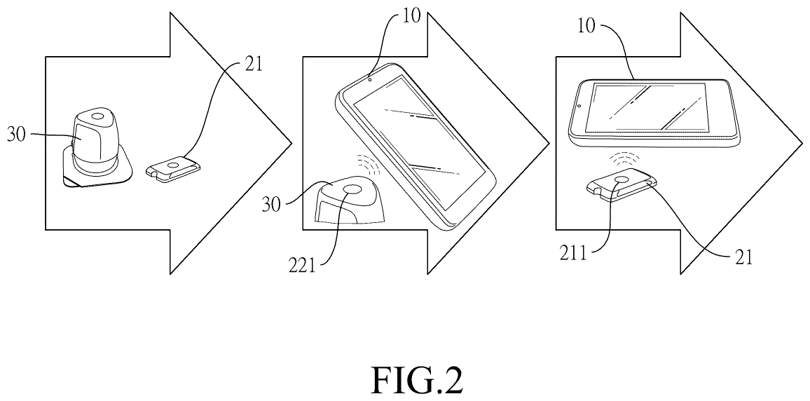

Please refer to FIG. 2, which is a schematic diagram of retrieving data through an information tag according to a specific embodiment of the present invention. In FIG. 2, the transmitter 21 (e.g., the transmitter 21 shown in FIG. 1A and FIG. 1B) of the present invention may include an information tag 211, which can be a near-field communication (NFC) tag or a barcode tag. The transmitter 21 is configured to store access information of the transmitter 21 to facilitate a pairing connection between the transmitter 21 and the handheld device 10. When the information tag 211 is the near-field communication (NFC) tag, the near-field communication (NFC) tag is configured on a circuit board inside the transmitter 21. When the information tag 211 is the barcode tag, the barcode tag is configured on a case of the transmitter 21 for the user to scan, wherein the barcode tag can be a two-dimensional barcode or a QR code, or any other pattern capable of storing information. The handheld device 10 (e.g., handheld device 10 shown in FIG. 1A and FIG. 1B) can obtain the access information of the transmitter 21 by magnetic field sensing the near-field communication (NFC) tag or scanning the barcode tag. The access information of the transmitter 21 may include, but is not limited to, a serial number of the transmitter and a media access control (MAC) address of a Bluetooth low energy device.

In addition, the implanting device 30 may include an information tag 221, and the information tag 221 can be a near-field communication (NFC) tag or a barcode tag, which is configured to store access information of the sensor 22 as shown in FIG. 1A and FIG. 1B. When the information tag 221 is the near-field communication (NFC) tag, the near-field communication (NFC) tag is configured inside a packaging case of the implanting device 30. When the information tag 221 is the barcode tag, the barcode tag is configured on the packaging case of the implanting device 30 for the user to scan, wherein the barcode tag can be a two-dimensional barcode or a QR code, or any other pattern capable of storing information. The handheld device 10 can obtain the access information of the sensor 22 by magnetic field sensing the near-field communication (NFC) tag or scanning the barcode tag. The access information of the sensor 22 may include, but is not limited to, a serial number, an expiration date and a parameter formula of the sensor.

In an information tag retrieving process, in a preparation stage, the user takes out the implanting device 30 with the information tag and the transmitter 21 with the information tag. Next, in a first data retrieving stage, the user obtains the information of the sensor 22 using the handheld device 10 to sense the near-field communication (NFC) tag or scan the barcode tag on the implanting device 30. Finally, in a second data retrieving stage, the user obtains the information of the transmitter 21 using the handheld device 10 to sense the near-field communication (NFC) tag or scan the barcode tag on the transmitter 21. After completing the information tag retrieving process, the handheld device 10 has obtained all the necessary information before installation. The sensor 22 is then implanted through the implanting device 30. After the sensor 22 is implanted on the epidermis of the user, the transmitter 21 is mounted on the sensor 22 to wirelessly transmit the analyte values measured by the sensor 22 to the handheld device 10 via the transmitter 21. In another embodiment, the user may first obtain the information of the transmitter 21, and then obtain the information of the sensor 22 through the handheld device 10. Specifically, in the first data acquisition stage after the preparation stage, the user obtains the information of the transmitter 21 using the handheld device 10 to sense the near-field communication (NFC) tag 211 or scanning the barcode label 211 on the transmitter 21. Then, in the second data acquisition stage, the user obtains the information of the sensor 22 using the handheld device 10 to sense the near-field communication (NFC) tag 221 or scanning the barcode label 221 on the implanting device 30.

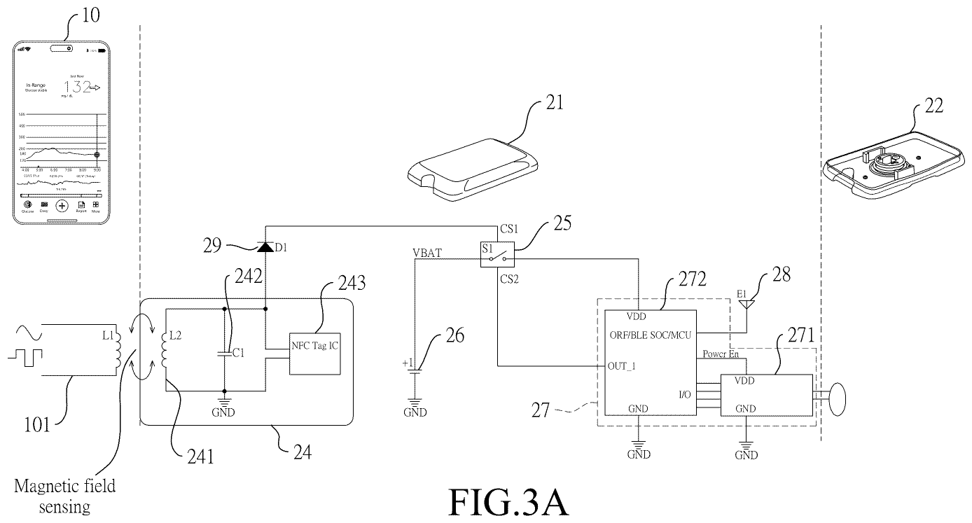

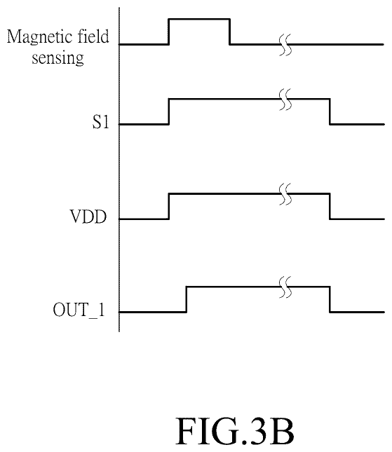

Please refer to FIG. 1A, FIG. 1B and FIG. 3A, wherein FIG. 3A is a circuit diagram of the handheld device 10 and the transmitter 21 according to a specific embodiment of the present invention. In FIG. 3A, the handheld device 10 includes a first inductive element 101. The transmitter 21 is coupled to the sensor 22, and includes: a magnetic field sensing unit 24, a switch unit 25, a power unit 26, a processing unit 27 and an antenna unit 28. The magnetic field sensing unit 24 is configured to supply power to the processing unit 27 in response to an inductive signal generated by an inductive magnetic field generated between the handheld device 10 and the magnetic field sensing unit 24. The magnetic field sensing unit 24 may include a second inductive element 241, a matching element 242 and an NFC chip 243, wherein the second inductive element 241 is a coil, and the matching element 242 is a capacitor. The second inductive element 241 is electrically connected to the matching element 242 in parallel to provide a low-resistance path for the inductive signal. The NFC chip 243 is configured to store the access information of the transmitter, such as the serial number and the media access control address of the transmitter. Therefore, the handheld device 10 generates the inductive magnetic field with the transmitter 21 through the interaction between the first inductive element 101 and the second inductive element 241, causing the magnetic field sensing unit 24 to generate the inductive signal through the inductive magnetic field to activate the processing unit 27, while the handheld device 10 obtains the access information of the NFC chip 243. Additionally, when the processing unit 27 is activated, the processing unit 27 performs a voltage detection of the power unit 26 to ensure that the power unit 26 has sufficient power.

The switch unit 25 is electrically connected to the magnetic field sensing unit 24, the power unit 26 and the processing unit 27, and includes a switch S1, a first control terminal CS1 and a second control terminal CS2. The processing unit 27 includes an analog-to-digital converter 271 and a microcontroller 272, wherein the analog-to-digital converter 271 is configured to receive and process a physiological signal measured by the sensor 22, and transmit the processed physiological signal to the microcontroller 272 to generate a processed signal. The first control terminal CS1 is coupled to a signal output terminal of the magnetic field sensing unit 24, and is configured to rectify the inductive signal generated by the magnetic field sensing unit 24 through a diode 29. The second control terminal CS2 is coupled to a signal output terminal OUT_1 of the microcontroller 272, and two ends of the switch S1 are coupled to the power unit 26 and a power terminal VDD of the microcontroller 272, respectively.

The antenna unit 28 is configured to transmit the processed signal to the handheld device 10. In the specific embodiment, the physiological signal processed by the microcontroller 272 is wirelessly transmitted to the handheld device 10 using a Bluetooth technology.

In a first operating cycle, the switch unit 25 is activated by the inductive signal, causing the power unit 26 to connect to the processing unit 27 through the switch unit 25, thereby activating the processing unit 27 to provide an activation signal to turn on the switch unit 25, so that the switch unit 25 enters a self-maintaining state through the activation signal. In a second operating cycle, the power unit 26 is connected to the processing unit 27 through the switch unit in the self-maintaining state to continuously supply power to the processing unit 27.

Please refer again to FIG. 3A and FIG. 3B to explain the implementation of the physiological signal sensing device 20. Once the handheld device 10 is brought close to the magnetic field sensing unit 24 in the physiological signal sensing device 20, the inductive magnetic field is generated, so as to generate the inductive signal and cause the inductive signal to trigger the switch S1 to turn on the switch unit 25. The turned-on switch unit 25 enables the power unit 26 to supply power to the power terminal VDD of the processing unit 27 to activate the processing unit 27. Next, the processing unit 27 provides the activation signal to the switch unit 25 through the signal output terminal OUT_1, causing the switch unit 25 to enter the self-maintaining state, which allows the power unit 26 to continuously supply power to the processing unit 27. During the period that the power unit 26 continuously supplies power to the processing unit 27, the processing unit 27 can continuously receive the physiological signal of the analyte measured by the sensor 22, generate the processed signal and transmit the processed signal to the antenna unit 28, and the antenna unit 28 wirelessly transmits the processed signal to the handheld device 10.

In this embodiment, the handheld device 10 and the magnetic field sensing unit 24 generate an inductive signal on the same frequency band. The inductive signal causes the switch S1 to be switched to the ON state through the first control terminal CS1, and thus, the power unit 26 can supply power to the processing unit 27 through the power terminal VDD of the microcontroller 272 to activate the microcontroller 272. The inductive signal can keep the switch S1 in the ON state until the inductive signal transitions to a low voltage state. Finally, the microcontroller 272 transmits a signal to the second control terminal CS2 of the switch S1 through an output terminal OUT_1 to maintain the ON state of the switch S1, so that the power unit 26 continues to supply power to the processing unit 27, thereby completing the self-maintaining control.

In a specific embodiment, the power terminal VDD of the analog-to-digital converter 271 is powered by a power supply terminal Power_EN of the microcontroller 272.

In a specific embodiment, the microcontroller may be a system-on-chip (SoC) that integrates radio frequency (RF) and/or Bluetooth Low Energy (BLE) wireless communications.

In practical applications, for example, the handheld device 10 can be a mobile phone or another signal-receiving device. In a specific embodiment, the physiological signal sensing device 20 can be a continuous glucose monitoring system, the sensor 22 can be partially implanted subcutaneously to measure a physiological signal of an analyte in a biological fluid, such as measuring a glucose concentration in a human body fluid. The measured physiological signal of the analyte can be processed through the analog-to-digital converter 271 and the processing unit 27, and then transmitted to the handheld device 10 through the antenna unit 28 wherein the measured physiological signal is calculated and calibrated by the handheld device 10 to obtain the blood glucose value. In the present invention, once the physiological signal sensing device 20 is activated, the switch unit 25 enters the self-maintaining state, so that the power unit 26 continues to supply power to the processing unit 27. The user can obtain the blood glucose values measured every minute through the user interface on the handheld device 10, and the blood glucose values can be formed as a blood glucose trend, thereby achieving functions such as continuous monitoring, trend analysis, abnormal alerts and historical data recording.

In a specific embodiment, the magnetic field between the handheld device 10 and the magnetic field sensing unit 24 is generated by energy of the same frequency band, wherein the frequency of the energy can be set to 13.56 MHz. However, the person having ordinary skill in the art will understand that the scope of the present invention is not limited to the above-mentioned frequency.

In a specific embodiment, the calculation and the calibration of the physiological signal can be performed by the analog-to-digital converter 271. The functions of the analog-to-digital converter can be implemented using hardware, software or firmware.

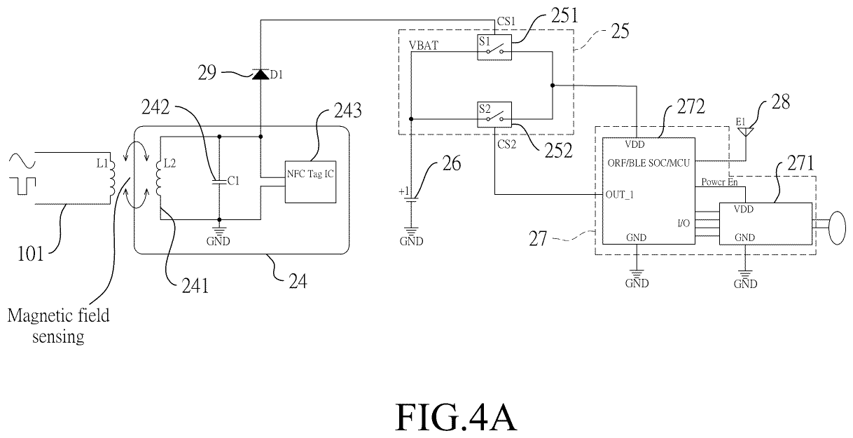

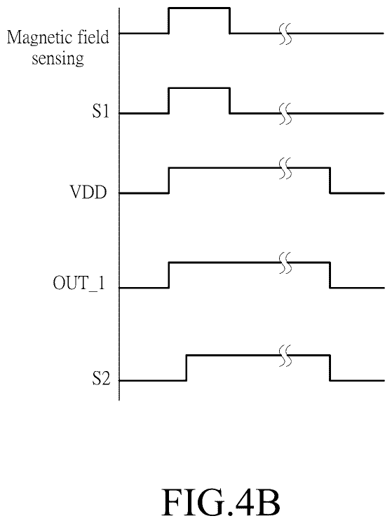

Please refer to FIG. 4A and FIG. 4B illustrating another specific embodiment of the physiological signal sensing device 20, wherein FIG. 4A is a system function block diagram of the handheld device 10 and the physiological signal sensing device 20, and FIG. 4B is a schematic diagram of timing state changes of each electronic parameter signal. In this embodiment, the switch unit 25 can include a first switch element 251 and a second switch element 252 connected in parallel, and the switch unit 25 is used to activate the processing unit 27 and complete the self-maintaining control.

In this embodiment, the handheld device 10 and the magnetic field sensing unit 24 first generates the inductive signal on the same frequency band. The inductive signal causes the switch S1 of the first switch element 251 to be switched to the ON state through the first control terminal CS1, so that the power unit 26 is connected to the processing unit 27 in the first operating cycle through the first switch element 251, and thus, the power unit 26 can supply power to the processing unit 27 through the power terminal VDD of the microcontroller 272 to activate the microcontroller 272. When the microcontroller 272 is activated, the signal is transmitted to the second control terminal CS2 through the output terminal OUT_1, causing the switch S2 of the second switch element 252 to be switched to the ON state. When the inductive signal transitions to a low voltage state, the switch S1 returns to the OFF state, but at this point, the switch S2 has already switched to the ON state. Therefore, in the second operating cycle, the power supply unit 26 continues to supply power to the processing unit 27 through the ON-stated second switch element 252, thereby completing the self-maintaining control.

In a specific embodiment, the antenna unit 28 wirelessly transmits the processed signal to the handheld device 10 using a Bluetooth communication technology. Specifically, when the antenna unit 28 transmits data to the handheld device 10, the distance between the handheld device 10 and the physiological signal sensing device 20 can be up to approximately 10 meters.

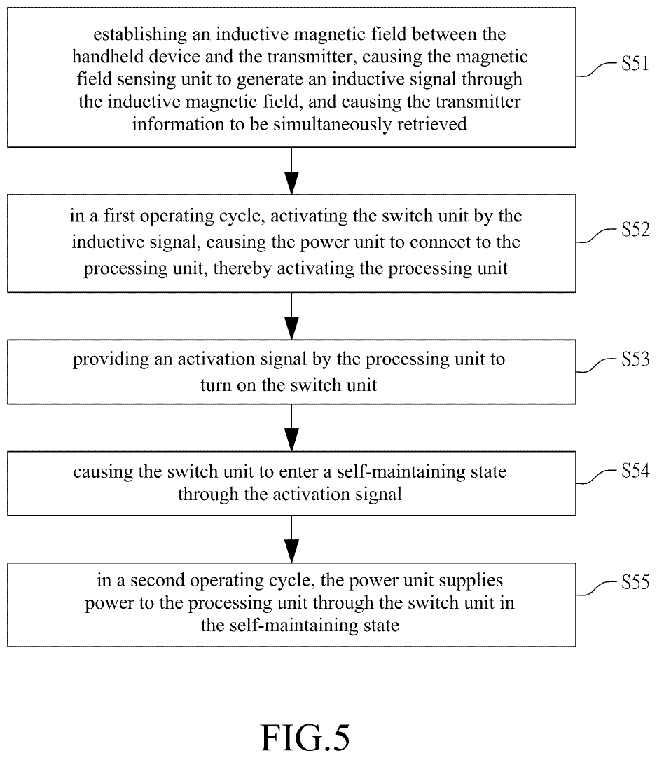

The present invention provides a method for activating the physiological signal sensing device 20 through the handheld device 10. The physiological signal sensing device 20 can includes the transmitter 21 and the sensor 22, wherein the sensor 22 is initially placed inside the implanting device 30 at the time of manufacturing. When in use, the sensor 22 is first implanted on the epidermis of a user through the implanting device 30, thereby a flexible needle is inserted into the subcutaneous tissue of the user's arm. The transmitter 21 is then mounted on the sensor 22 to wirelessly transmit analyte values measured by the sensor 22 to the handheld device 10. Based on the data retrieving process in FIG. 2 and the embodiment of activating the power of the transmitter 21 in FIG. 3A, please refer to a transmitter activation flow chart in FIG. 5 to illustrate the implementation process. In step S51, the inductive magnetic field between the handheld device 10 and the transmitter 21 is established, causing the magnetic field sensing unit 24 to generate the inductive signal through the inductive magnetic field, and causing the transmitter information stored in the NFC chip 243 to be simultaneously retrieved, wherein the transmitter information includes the serial number of the transmitter and the media access control (MAC) address of the Bluetooth low energy device. In step S52, in the first operating cycle, the switch unit 25 is activated by the inductive signal, causing the power unit 26 to connect to the processing unit 27 through the switch unit 25, thereby activating the processing unit 27 and allowing the transmitter 21 to begin operation. Next, in step S53, the activation signal is provided by the processing unit 27 to turn on the switch unit 25. Then, in step S54, the switch unit 25 is entered the self-maintaining state through the activation signal. In step S55, in the second operating cycle, the power unit 26 is connected to the processing unit 27 through the switch unit 25 in the self-maintaining state to supply power to the processing unit 27.

The method for activating the physiological signal sensing device 20 through the handheld device 10 in the embodiments of the present invention allows the user to simultaneously complete the necessary information retrieval and power activation through convenient magnetic field sensing or code scanning methods, which effectively simplifies the installation steps for the physiological signal sensing device 20, thereby optimizing the user experience. In the embodiment of the present invention, the self-maintaining state of the switching unit 25 is completed through the timing coordination of the inductive signal and the activation signal, ensuring that the power unit 26 continuously supplies power to the processing unit 27 without interruption during both the first operating cycle and the second operating. Therefore, through the method for activating the physiological signal sensing device 20 in the present invention, the purpose of the transmitter 21 successfully wirelessly transmitting the analyte values measured by the sensor 22 to the handheld device 10 is achieved, whereby the user can obtain the blood glucose-related information (such as blood glucose trend, current blood glucose levels, historical blood glucose levels, etc.) through the user interface of the handheld device 10 to monitor the blood glucose over a full cycle.

Although the present invention has been described with reference to certain exemplary embodiments thereof, it can be understood by those skilled in the art that a variety of modifications and variations may be made to the present invention without departing from the spirit or scope of the present invention defined in the appended claims, and their equivalents.

1. A physiological signal sensing device activated through a handheld device, comprising:
a sensor configured to be partially implanted subcutaneously to measure a physiological signal of an analyte in a biological fluid; and
a transmitter coupled to the sensor, comprising:
a magnetic field sensing unit capable of generating an inductive magnetic field with the handheld device at a predetermined distance to generate an inductive signal;
a switch unit connected to the magnetic field sensing unit;
a power unit connected to the switch unit;
a processing unit connected to the switch unit and configured to receive and process the physiological signal measured by the sensor to generate a processed signal; and
an antenna unit connected to the processing unit and configured to receive the processed signal and transmit the processed signal to the handheld device;
wherein in a first operating cycle, the switch unit is activated by the inductive signal, causing the power unit to connect to the processing unit through the switch unit, thereby activating the processing unit to provide an activation signal to turn on the switch unit, causing the switch unit to enter a self-maintaining state through the activation signal; and
in a second operating cycle, the power unit connects to the processing unit through the switch unit in the self-maintaining state to supply power to the processing unit.

2. The physiological signal sensing device as claimed in claim 1, wherein the inductive magnetic field between the handheld device and the magnetic field sensing unit is generated by energy of the same frequency band.

3. The physiological signal sensing device as claimed in claim 1, wherein the switch unit comprises a first switch element and a second switch element connected in parallel, such that in the first operating cycle, the first switch element is turned on by the inductive signal, causing the power unit to connect to the processing unit through the first switch element, thereby activating the processing unit to provide the activation signal to activate the second switch element, causing the second switch element to enter the self-maintaining state through the activation signal; and in the second operating cycle, the first switch element is turned off and the second switch element is turned on, causing the power unit to connect to the processing unit through the second switch element to supply power to the processing unit.

4. The physiological signal sensing device as claimed in claim 1, further comprising a near-field communication (NFC) tag installed on the transmitter and configured to store access information of the transmitter, wherein the handheld device obtains the access information of the transmitter through inductive magnetic field with the magnetic field sensing unit, and the access information of the transmitter comprises a serial number and a media access control (MAC) address of the transmitter.

5. The physiological signal sensing device as claimed in claim 1, further comprising a barcode tag installed on the transmitter and configured to store access information of the transmitter, wherein the handheld device obtains the access information of the transmitter by scanning the barcode tag, and the access information of the transmitter comprises a serial number and a media access control (MAC) address of the transmitter.

6. The physiological signal sensing device as claimed in claim 1, further comprising a near-field communication (NFC) tag installed inside a packaging case of the sensor and configured to store access information of the sensor, wherein the handheld device obtains the access information of the sensor through inductive magnetic field with the NFC tag, and the access information of the sensor comprises a serial number, an expiration date and a parameter formula of the sensor.

7. The physiological signal sensing device as claimed in claim 1, further comprising a barcode tag installed on a packaging case of the sensor and configured to store access information of the sensor, wherein the handheld device obtains the access information of the sensor by scanning the barcode tag and the access information of the sensor comprises a serial number, an expiration date and a parameter formula of the sensor.

8. A method for activating a physiological signal sensing device through a handheld device, wherein the physiological signal sensing device is configured to be partially implanted subcutaneously to measure a physiological signal of an analyte in a biological fluid and comprises a transmitter and a sensor, and the transmitter comprises a power unit, a switch unit, a magnetic field sensing unit and a processing unit, the method comprising:
establishing an inductive magnetic field between the handheld device and the transmitter at a predetermined distance, causing the magnetic field sensing unit to generate an inductive signal through the inductive magnetic field;
in a first operating cycle, activating the switch unit by the inductive signal, causing the power unit to connect to the processing unit through the switch unit, thereby activating the processing unit;
providing an activation signal by the processing unit to turn on the switch unit;
causing the switch unit to enter a self-maintaining state through the activation signal; and
in a second operating cycle, connecting the power unit to the processing unit through the switch unit in the self-maintaining state to supply power to the processing unit.

9. The method as claimed in claim 8, wherein the inductive magnetic field between the handheld device and the magnetic field sensing unit is generated by providing energy of the same frequency band.

10. The method as claimed in claim 8, wherein the switch unit comprising a first switch element and a second switch element connected in parallel, and the method further comprises, in the first operating cycle, activating the first switch element by the inductive signal, causing the power unit to connect to the processing unit through the first switch element, thereby activating the processing unit to provide the activation signal to turn on the second switch element, causing the second switch element to enter the self-maintaining state through the activation signal; and in the second operating cycle, turning off the first switch element and turning on the second switch element, causing the power unit to connect to the processing unit through the second switch element to supply power to the processing unit.

11. The method as claimed in claim 8, wherein, while establishing the inductive magnetic field between the handheld device and the transmitter, the method further comprises:
obtaining access information of the transmitter using the handheld device, wherein the access information of the transmitter is stored in a near-field communication (NFC) tag, and the access information of the transmitter comprises a serial number and a media access control (MAC) address of the transmitter.

12. The method of as claimed in claim 8, further comprising:
obtaining access information of the sensor using the handheld device, wherein the access information of the sensor is stored in a near-field communication (NFC) tag, and the access information of the sensor comprises a serial number, an expiration date, a parameter formula of the sensor.

13. The method as claimed in claim 8, further comprising detecting a voltage of the power unit using the processing unit.

14. A method for activating a physiological signal sensing device through a handheld device, wherein the physiological signal sensing device is configured to be partially implanted subcutaneously to measure a physiological signal of an analyte in a biological fluid and comprises a transmitter and a sensor, and the transmitter comprises a power unit, a switch unit, a magnetic field sensing unit and a processing unit, the method comprising:
scanning a barcode tag of the transmitter using the handheld device to obtain access information of the transmitter;
scanning a barcode tag of the sensor using the handheld device to obtain access information of the sensor;
establishing an inductive magnetic field between the handheld device and the transmitter at a predetermined distance, causing the magnetic field sensing unit to generate an inductive signal through the inductive magnetic field;
in a first operating cycle, turning on the switch unit by the inductive signal, causing the power unit to connect to the processing unit through the switch unit to activate the processing unit;
providing an activation signal by the processing unit to turn on the switch unit;
causing the switch unit to enter a self-maintaining state through the activation signal; and
in a second operating cycle, connecting the power unit to the processing unit through the switch unit in the self-maintaining state to supply power to the processing unit.

15. The method as claimed in claim 14, wherein the inductive magnetic field between the handheld device and the magnetic field sensing unit is generated by providing energy of the same frequency band.

16. The method as claimed in claim 14, wherein the switch unit comprising a first switch element and a second switch element connected in parallel and the method further comprises in the first operating cycle, turning on the first switch element by the inductive signal, causing the power unit to connect to the processing unit through the first switch element, thereby activating the processing unit to provide the activation signal to turn on the second switch element, causing the second switch element to enter the self-maintaining state through the activation signal; and in the second operating cycle, turning off the first switch element and turning on the second switch element, causing the power unit to connect to the processing unit through the second switch element to supply power to the processing unit.

17. The method as claimed in claim 14, wherein the access information of the transmitter comprises a serial number and a media access control (MAC) address of the transmitter.

18. The method as claimed in claim 14, wherein the access information of the sensor comprises a serial number, an expiration date and a parameter formula of the sensor.

19. The method as claimed in claim 14, further comprising detecting a voltage of the power unit using the processing unit.
